(19) United States
(12) Patent Application Publication
(10) Pub. No.: US 2022/0266056 A1
(43) Pub. Date: Aug. 25, 2022

(54) Light Therapy System

(71) Applicant: Kiierr International, LLC, Salt Lake City, UT (US)

(72) Inventors: Raymond R. Blanche, Chatham, NJ (US); Michael Anderson, Butte, MT (US)

(21) Appl. No.: 17/667,944

(22) Filed: Feb. 9, 2022

Related U.S. Application Data
(60) Provisional application No. 63/153,186, filed on Feb. 24, 2021.

(51) Int. Cl. A61N 5/06; A61N 5/067

(52) U.S. Cl. CPC A61N 5/0617; A61N 5/067; A61N 2005/0632; A61N 2005/0647; A61N 2005/0652; A61N 2005/0662

(57) ABSTRACT

A plurality of light emitting components are disclosed disposed about a flexible substrate shaped to approximate a portion of the human body. The flexible substrate has a plurality of slots that allow portions of the substrate to flex to accommodate different shaped portions of the human body. A shell is disposed atop the flexible substrate and is biased in a first position to have an internal volume. The shell has a plurality of slots allowing portions of the shell to flex to increase or decrease the internal volume of the shell.

### CLAIM OF PRIORITY

The present application claims priority to U.S. Ser. No. 63/153,186 entitled “Light Therapy System” filed on Feb. 24, 2021 which is incorporated herein by reference in its entirety.

### FIELD OF THE TECHNOLOGY

The present technology relates generally to light therapy devices, systems, and associated methods and more particularly to an improved apparatus and system for providing light therapy intended to stimulate hair growth, among other things.

### BACKGROUND

Hair loss, and its accompanying problems, are common and may be the cause of personal concern for many. Treatment of hair loss includes topical creams or medications, ingestible medications, and surgical treatment. These methods are expensive and time consuming. Light therapy has been used to stimulate hair growth. However, apparatuses and methods of such therapy are difficult to adapt to the different types of problems or areas of the body to be treated. It is therefore desirable to have improved methods and apparatuses for promoting the growth of hair or other issues related to dermal tissue.

### BRIEF DESCRIPTION OF THE DRAWINGS

To further clarify the above and other aspects of the present technology, a more particular description of the invention will be rendered by reference to specific aspects thereof which are illustrated in the appended drawings. It is appreciated that these drawings depict only typical aspects of the technology and are therefore not to be considered limiting of its scope. The drawings are not drawn to scale. The technology will be described and explained with additional specificity and detail through the use of the accompanying drawings in which:

FIG. 1 is a front perspective view of a lighting device in accordance with one aspect of the technology;

FIG. 2 is a top view of the lighting device of FIG. 1;

FIG. 3 is a back perspective view of the lighting device of FIG. 1;

FIG. 4 is a flexible printed circuit board in accordance with one aspect of the technology;

FIG. 5 is a view of the lighting device of FIG. 1 in accordance with one aspect of the technology;

FIG. 6 is a view of the lighting device of FIG. 1 in accordance with one aspect of the technology;

FIG. 7 is a view of the lighting device of FIG. 1 in accordance with one aspect of the technology;

FIG. 8 is a view of the lighting device of FIG. 1 in accordance with one aspect of the technology;

FIG. 9 is a top view of a flexible printed circuit board in accordance with one aspect of the technology; and

FIG. 10 is a top view of a flexible mesh in accordance with one aspect of the technology.

### DESCRIPTION OF TECHNOLOGY

Although the following detailed description contains many specifics for the purpose of illustration, a person of ordinary skill in the art will appreciate that many variations and alterations to the following details can be made and are considered to be included herein. Accordingly, the following aspects are set forth without any loss of generality to, and without imposing limitations upon, any claims set forth. It is also to be understood that the terminology used herein is for the purpose of describing particular aspects only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a layer” includes a plurality of such layers.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the aspects described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the aspects described herein are, for example, capable of operation in other orientations than those illustrated or otherwise described herein. The term “coupled,” as used herein, is defined as directly or indirectly connected in an electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.”

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one aspect. Thus, appearances of the phrases “in an example” in various places throughout this specification are not necessarily all referring to the same aspect.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed aspects and that no equality or consistency in the amount, degree, or realization of improved performance is to be assumed as universally applicable.

### Example Aspects

An initial overview of technology aspects are provided below and specific technology aspects are then described in further detail. This initial summary is intended to aid readers in understanding the technology more quickly, but is not intended to identify key or essential features of the technology, nor is it intended to limit the scope of the claimed subject matter.

Broadly speaking, aspects of the disclosed technology create a unique and improved utilitarian light, configured and equipped with an adjustable shape to accommodate different shaped portions of the human body, including for example, different shaped heads. In one aspect of the technology, a lighting device comprises a flexible printed circuit board (or PCB) with a plurality of light emitting diodes (LEDs) and/or lasers disposed thereon. The PCB is coupled to a flexible outer shell on a first, exterior side of the PCB, and a flexible inner shell on a second, interior side. In one aspect, the outer shell is opaque or transparent. However, the inner shell is transparent so that light from the LEDs and/or lasers may pass through and onto the scalp of the user. Each of the PCB, inner shell, and outer shell comprise a plurality of curvilinear slots, cuts, or voids so that they may each be shaped to approximate a human skull. In any event, the PCB, inner shell, and outer shell, are mated together so that the three create a deformable cap that can be worn by many different users. In one aspect, the PCB and the inner shell comprise an integrated unit or assembly such that the device comprises an inner shell (which houses or is integrated with the PCB) and an outer shell.

In other aspects of the technology, the outer shell comprises an air channel disposed about a top of the outer shell. The air channel extends from a back portion of the outer shell to a front portion of the outer shell. The depth of the channel decreases as the channel extends from the back of the outer shell to the front. The air channel allows for the device to be cooled during use.

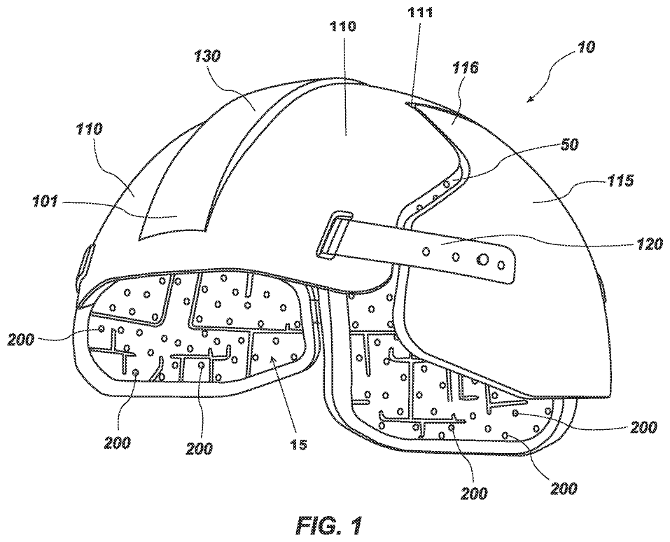

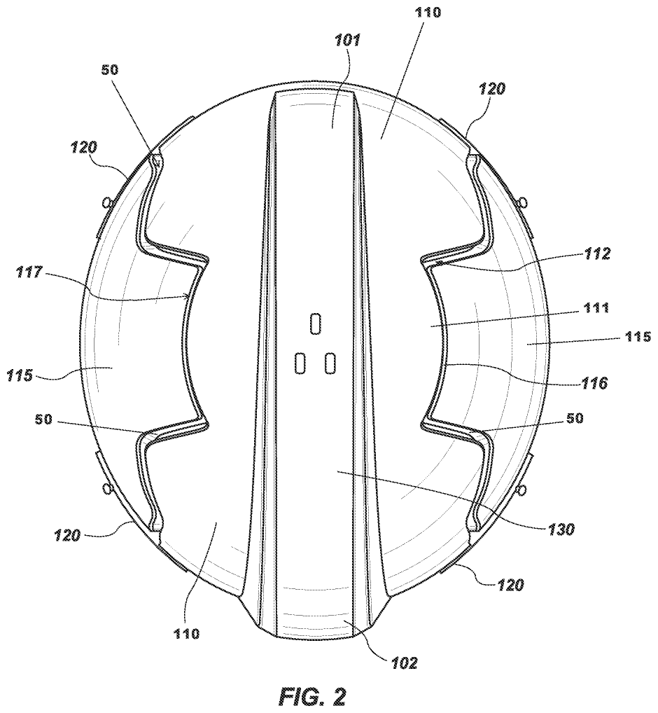

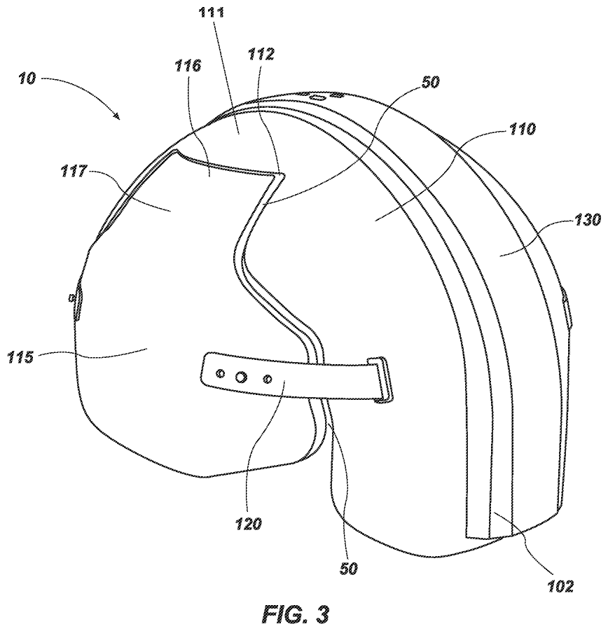

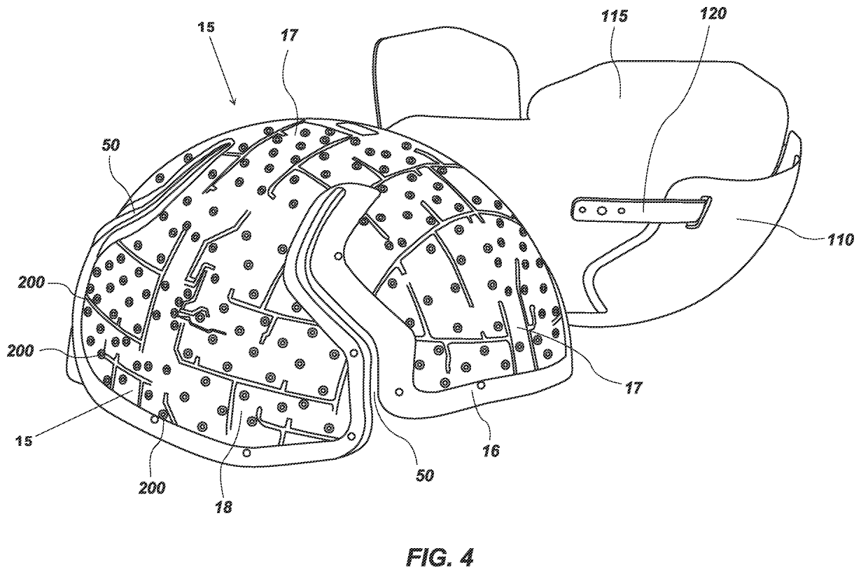

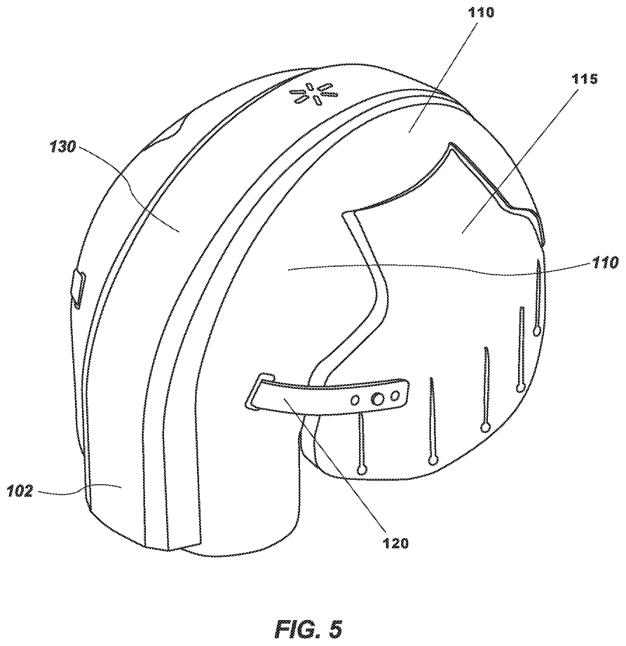

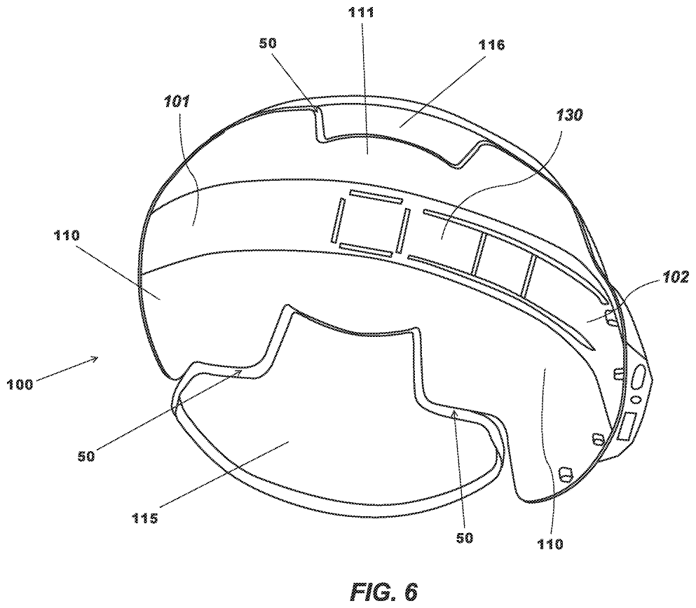

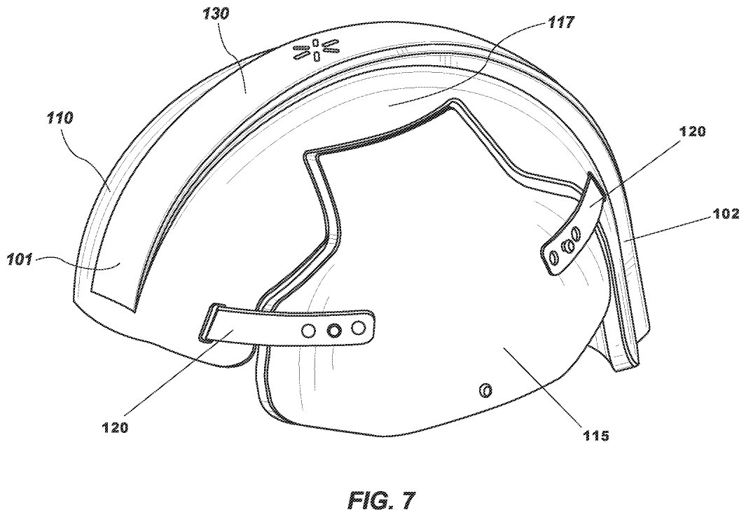

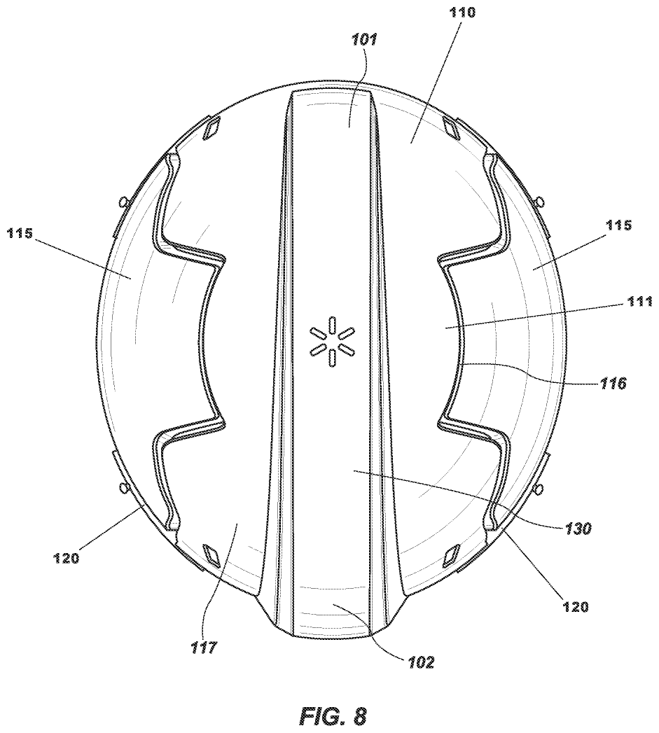

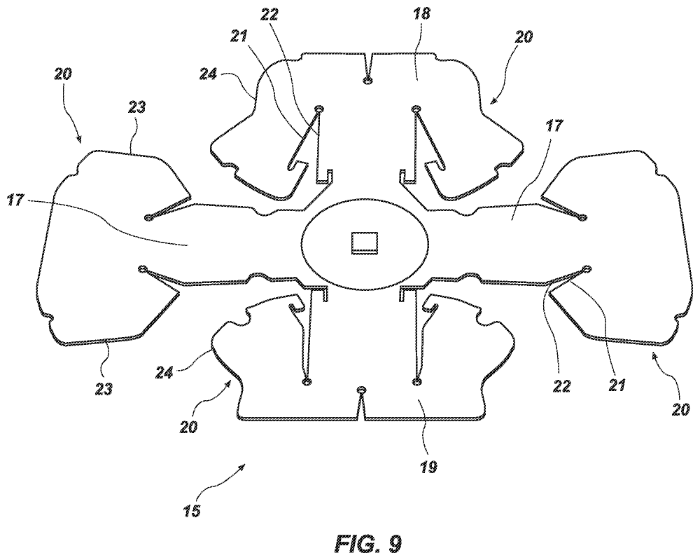

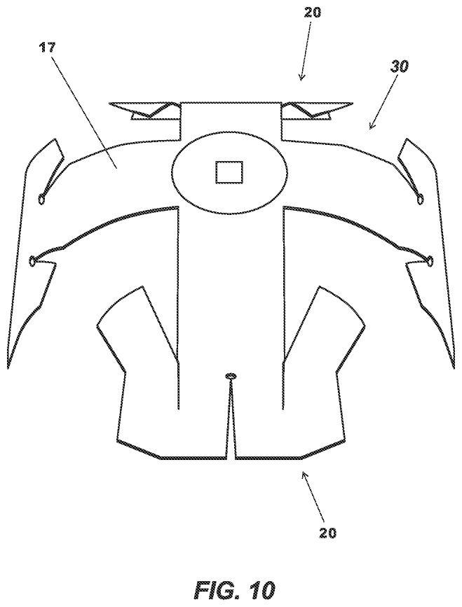

With reference specifically to FIGS. 1-10, a light therapy device 10 comprises a flexible substrate (e.g., a PCB, etc.) 15 with a plurality of light sources disposed thereon. In one aspect, the light sources comprise between 150 and 250 LEDs 200. The PCB 15 is contoured so that when it is shaped to approximate a dome, it comprises a plurality of curvilinear slots 50 configured to assist the user in “fine-tuning” the shape of the PCB 15 to better approximate individual human heads. However, the PCB 15 may also be manipulated into different shapes as well without departing from the scope of the technology. For example, in one aspect, the PCB 15 is configured with curvilinear slots that allow a user to shape the PCB 15 into an oval to approximate a portion of an arm or leg that may benefit from light therapy.

In one aspect of the technology, the PCB 15 comprises a center component 17 and two side flaps 18, 19 on opposing sides of the center component 17. When in a planar configuration, the PCB is generally cross-shaped with protruding ends 20 about each end of the cross. On each of the protruding ends 20, the PCB is cut or shaped so that when it is formed into a curvilinear shape (e.g., dome shaped, oval shaped, etc.) the internal edge 21 folds into (or defines) an adjacent internal edge 22. When folded (or otherwise placed) together, exterior edge 23 and exterior edge 24 are adjacent, but are not coupled together. Rather, they form slots or openings 50 which allow the PCB 15 to flex to accommodate different shaped treatment targets (e.g., different shaped heads, legs, etc.). In one aspect, the PCB 15 is coupled to a flexible mesh 30 to approximate the shape of the PCB 15.

In one aspect of the technology, the PCB 15 comprises a flexible casing or shell 16 disposed about the PCB 15. The casing is made from a plastic, thermoplastic, or similar materials. The casing or shell 16 comprises a plurality of slots that are shaped to approximate the slots 50 disposed about the PCB 15. The casing 16 is transparent at least on the side of the PCB 15 comprising the LEDs 200 to permit the propagation of light therethrough. The flexible casing 16 is generally shaped to approximate the shape of the PCB 15, but is slighting larger so that it encompasses the PCB therein. When the PCB 15 and casing 16 are shaped to approximate a portion of the human body (e.g., a top of the skull), they retain the slots 50 therein to assist in flexing about the shape of the body to accommodate a better final fit. In one aspect the PCB 15 and casing 16 comprise an inner shell or lighting sub assembly.

In one aspect of the technology, the PCB 15 and flexible casing 16 are covered by a rigid, semi-rigid, or flexible cap or outer shell 100. The cap or outer shell 100 comprises a plastic, thermoplastic, rubber, or other material. In one aspect of the technology, the outer shell or cap 100 is configured such that it is biased in a first (or resting) position defining a first internal volume where the head (or other body part) of the user resides during use. In this aspect, the first position represents the smallest internal volume during regular use of the device. In one aspect, the outer shell or cap 100 may be flexed in order to reside in a second position. The second position comprises a larger internal volume than the first position which can accommodate a larger head (or other body part) therein. The outer shell or cap 100 may also be flexed to have third, fourth, and fifth positions with increasingly larger internal volumes. In another aspect, the outer shell or cap 100 is biased in a position wherein its largest internal volume is the resting position. In this aspect, the internal volume of the outer shell or cap 100 is adjusted so that the internal volume is incrementally smaller rather than incrementally larger. In either aspect, the inner shell and PCB (or inner shell and PCB assembly) are configured to flex in a similar proportion as the outer shell or cap 100. However, in one aspect, the inner shell 16 and PCB 15 are not biased in a particular position.

The cap or outer shell 100 comprises a center component 110 configured to extend from the back of the neck of the user, forward to the forehead of the user. The cap or outer shell 100 also comprises two side flaps or lateral members 115 disposed on opposing sides of the center component 110. The center component 110 is coupled to the side flaps (or lateral members) 115 between a side edge 111 of the center component and a top edge 116 of the side flaps. The sides and bottom of the side flaps 115 are moveable with respect to the center component 110, pivoting or flexing about the top edge 116. In this manner, different-sized heads (or other body parts) may be accommodated by a single cap 100. In one aspect, the side flaps or lateral members 115 are configured such that they may flex inward or outward with respect to the center component 110 ranging from 0.5 to 1.5 inches without suffering plastic deformation or breaking.

While a center component 110 extends from the back of the head to the forehead of the user with flexible side flaps 115, it is understood that in other aspects of the technology, the center component extends from side to side with flexible flaps on the back and front portion of the outer shell or cap 100.

Generally speaking, the center component 110 comprises a recess 112 configured to receive a top portion 117 of the side flap therein. In one aspect of the technology, the side flaps 115 are coupled to an adjacent portion of the center component 110 by way of one or more straps 120 or other securement device. The strap 120 is adjustable to allow the side flaps 115 to move outward and away from the head (or other body part) of the user, as suits a particular head (or other body part). The strap 120 comprises a plurality of holes configured to mate with a tab, peg, or pin disposed about the side flap 115. The tab, peg, or pin can be located in a number of different locations to accommodate a number of different traps, including near the chin of the user. In this manner, the center component 110 may also flex outward about its longitudinal axis to accommodate rearward and frontward differences in head (or other body part) size or shape. In one aspect, the straps 120 are flexible and are configured to keep the side flaps 115 from being over extended. In another aspect, the straps 120 are rigid or semi-rigid such that they keep the side flaps 115 in whatever position they are secured into when placed in a particular hole within the straps 120. While a strap 120 with holes is disclosed herein, it is understood that any securement device could be used in connection with the subject device without departing from the objection of the technology. For example, a post or brace may be used to secure the side flaps 115 in a particular position.

In one aspect of the technology, the side flaps 115 are configured to extend downward about the sides of the head of the user further than a front of the center component 110. A back of the center component extends downward further than the side flaps 115. In one aspect, a center of the center component 110 of the cap (or other shell) 100 comprises an air channel 130 configured to conduct heat away from the head (or other body part) of the user. In one aspect, the air channel 130 comprises one or more mechanical fans coupled to the power source for pushing or pulling air through the air channel 130. The air channel 130 comprises a first depth near the front 101 of the cap 100 gradually increasing to a second depth near the back 102 of the cap 100. In this manner, the amount of air flow through the air channel 130 is optimized and a maximum amount of air flow is conducted through the air channel 130 while minimizing the size of the air channel 130 about the top of the cap 100. In one aspect a plurality of vent holes are disposed about a top of the air channel 130 to provide supplemental air flow through the air channel 130 and/or to provide and additional vent for heat when the fan is not activated. In one aspect, the width of the air channel 130 is substantially the same from the front 101 of the cap 100 to the back 102 of the cap 100. However, in another aspect, the width of the air channel gradually increases from the front 101 to the back 102 of the cap 100.

In one aspect of the technology, the PCB 15 comprises, or is coupled to, a programmable logic circuit (PLC) that regulates power and control of the light source (including but not limited to LEDs and/or lasers). In one aspect of the technology, the PLC is configured to regulate the pulse-width-modulation (or PWM) of the LEDs 200 at a plurality of different duty cycles in a plurality of different sequences. PWM is one way of regulating the brightness of a light. In one aspect, light emission from the LED 200 is controlled by pulses wherein the width of these pulses is modulated to control the amount of light perceived by the user of the lighting device. When the full direct current voltage runs through an LED, the maximum of light is emitted 100% of the time. That is, the LED 200 emits light 100% of the time when in an “ON” mode. With PWM, the voltage supplied to the LED 200 can be “ON” 50% of the time and “OFF” 50% of the time so that the LED 200 gives off its maximum amount of light only 50% of the time. This is referred to as a 50% duty cycle. In this scenario, if the ON-OFF cycle is modulated fast enough, human eyes will perceive only half the amount of light coming from the LED 200. That is, with such an input on the LED 200, the amount of light given off appears diminished by 50%. While specific reference is made to a 50% duty cycle, the LED 200 duty cycle of the light sources described herein may be greater or lesser than 50% as suits a particular purpose. In one aspect, the PLC, power source, a control switch, and different light sources are all operably coupled together.

In one aspect of the technology, different light sequencing modes are programmed into the PLC. In one aspect of the technology, the PWM for the HIGH, MEDIUM, and LOW modes (or first, second, and third power modes) is set at a duty cycle of about 75%, 50%, and 25%, respectively, though other duty cycles may be used. In one aspect, the duty cycle for the first power mode ranges from about 70% to 80%, the second power mode ranges from about 45% to 55%, and the third power mode ranges from about 20% to 30%. The duty cycles may alternate or may be timed to be ON for a predetermined period of time and OFF for a predetermined period of time. For example, in one aspect the LEDs are powered ON for 0.5 seconds and powered OFF for 0.5 seconds in an alternating sequence.

It is noted that one or more sets or subsets of LEDs 200 may be activated to have different duty cycles as suits a particular application. For example, a subset of LEDs that are configured to target the top of the scalp that may have more hair loss may have a duty cycle of 75% while a subset of LEDs that are configured to target the side of the scalp with less hair loss may have a duty cycle of 25%. Other duty cycles are of course contemplated herein. In one aspect of the technology, the duty cycle of respective portions of the lighting device may be adjusted by the user. In this manner, the user may designate specific areas where hair loss is more or less problematic and efficiently direct more or less light to that area of the body. Advantageously, less energy is used to accomplish the goals of the end user.

In addition to different duty cycles, in one aspect of the technology, the LEDs 200 are configured to propagate different wavelengths of light. In one aspect, the first plurality of LEDs are configured to propagate light at a wavelength ranging from about 640 nm to 660 nm. A second plurality of LEDs are configured to propagate light at a wavelength ranging from about 930 nm to 950 nm. In one aspect, the first plurality of LEDs comprises between 50 and 150 LEDs and the second plurality of LEDs comprises between 50 and 150 LEDs. In one aspect of the technology, the first and second plurality of LEDs simultaneously propagate light onto the scalp (or other area of skin) while in another aspect of the technology, the first and second plurality of LEDs alternate between ON and OFF modes such that the first plurality of LEDs and the second plurality of LEDs are not on at the same time.

It is noted that no specific order is required in these methods unless required by any claims set forth herein, though generally in some aspects, the method steps can be carried out sequentially.

The foregoing detailed description describes the technology with reference to specific exemplary aspects. However, it will be appreciated that various modifications and changes can be made without departing from the scope of the present technology as set forth in the appended claims. The detailed description and accompanying drawing are to be regarded as merely illustrative, rather than as restrictive, and all such modifications or changes, if any, are intended to fall within the scope of the present technology as described and set forth herein.

More specifically, while illustrative exemplary aspects of the technology have been described herein, the present technology is not limited to these aspects, but includes any and all aspects having modifications, omissions, combinations (e.g., of aspects across various aspects), adaptations and/or alterations as would be appreciated by those skilled in the art based on the foregoing detailed description. The limitations in the claims are to be interpreted broadly based on the language employed in the claims and not limited to examples described in the foregoing detailed description or during the prosecution of the application, which examples are to be construed as non-exclusive. For example, in the present disclosure, the term “preferably” is non-exclusive where it is intended to mean “preferably, but not limited to.” Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus-function are expressly recited in the description herein. Accordingly, the scope of the invention should be determined solely by the appended claims and their legal equivalents, rather than by the descriptions and examples given above.

1. A portable lighting device for stimulating hair growth, comprising:
a plurality of light emitting components disposed about a flexible substrate shaped to approximate a portion of the human body, said flexible substrate comprising a plurality of slots configured to allow portions of the flexible substrate to flex to accommodate different shaped portions of the human body; and
a shell disposed atop the flexible substrate, said shell biased in a first position have an internal volume, said shell comprising a plurality of slots allowing portions of the shell to flex to increase or decrease the internal volume of the shell.

2. The lighting device of claim 1, wherein the shell comprises a center portion and a plurality of opposing flexible side flaps.

3. The lighting device of claim 2, wherein the center portion extends about a longitudinal length from a front portion to a back portion forming a non-linear shape.

4. The lighting device of claim 3, wherein the side flaps extend downward from the center portion to a position below the front portion and above the back portion.

5. The lighting device of claim 2, wherein the center portion comprises a channel disposed about a longitudinal length of the center portion.

6. The lighting device of claim 5, wherein the channel comprises a first depth about the front of the center portion and a second depth about the back of the center portion.

7. The lighting device of claim 6, wherein the depth of the channel gradually increases from the front of the center portion to the back of the center portion.

8. A portable and wearable lighting device for stimulating hair growth, comprising:
a plurality of light emitting components disposed about a substrate arranged in a curvilinear shape defining an internal volume, said substrate comprising a plurality of slots configured to allow portions of the substrate to flex thereby increasing or decreasing the internal volume;
a shell disposed about an exterior of the plurality of light emitting components, said shell comprising a plurality of slots configured to allow portions of the shell to flex to define a plurality of internal volumes of the shell; and
a programmable logic controller configured to regulate power and control of the light emitting components.

9. The lighting device of claim 8, wherein the plurality of light emitting components comprise a duty cycle ranging from 25% to 75%.

10. The lighting device of claim 8, wherein the plurality of light emitting components comprise LEDs or lasers.

11. The lighting device of claim 8, wherein the plurality of light emitting components comprise a first portion of light emitting components and a second portion of light emitting components, the first portion being located in a first area of the substrate and the second portion being located in a second area of the substrate, the first area being different than the second area.

12. The lighting device of claim 11, wherein the duty cycle of the first portion of the plurality of light emitting components is different than the duty cycle of the second portion of the plurality of light emitting components.

13. The lighting device of claim 11, wherein power is propagated alternately between the first portion of the plurality of light emitting components and the second portion of the plurality of light emitting components at a predetermined time period.

14. The lighting device of claim 8, wherein the plurality of light emitting components propagate light at wavelengths ranging from 640 nm to 660 nm and 930 nm to 950 nm.

15. The lighting device of claim 8, wherein the plurality of light emitting components comprises a first plurality of LEDs configured to propagate light at a wavelength ranging from 640 nm to 660 nm and a second plurality of LEDs configured to propagate light at a wavelength ranging from 930 nm to 950 nm.

16. A method of stimulating hair growth, comprising:
placing a lighting device about a portion of a body;
said lighting device comprising a substrate with a plurality of LEDs, the substrate having a curvilinear shape defining an internal volume, said substrate comprising a plurality of slots configured to allow portions of the substrate to flex to define a plurality of internal volume, and a shell disposed about an exterior of the plurality of light emitting components, said shell comprising a plurality of slots configured to allow portions of the shell to flex to define a plurality of internal volumes of the shell; and
propagating light at a predetermined wavelength from the lighting device to the portion of the body.

17. The method of claim 16, wherein the shell comprises a rigid center component extending from a front of the shell to a back of the shell and side flaps flexibly coupled to the rigid center component.

18. The method of claim 16, wherein the plurality of LEDs comprises a first portion of LEDs and a second portion of LEDs.

19. The method of claim 18, further comprising propagating light from the first portion of LEDs at a first duty cycle and propagating light from the second portion of LEDs at a second duty cycle, wherein the first duty cycle is different than the second duty cycle.

20. The method of claim 18, further comprising propagating light from the first portion of LEDs at a wavelength of light ranging from 640 nm to 660 nm and from the second portion of LEDs at a wavelength of light ranging from 930 nm to 950 nm.
